(19) United States
(12) Patent Application Publication
(10) Pub. No.: US 2019/0008199 A1
(43) Pub. Date: Jan. 10, 2019

(54) PROTEIN-RICH ENTERAL NUTRITIONAL COMPOSITION CONTAINING A HIGH PROPORTION OF CASEINATES

(71) Applicant: EVEN SANTE INDUSTRIE, Lesneven (FR)

(72) Inventors: Audrey LE FUR, Bourg Blanc (FR); Jean-Marc LE PALUD, Plabennec (FR); Anne FOSSEUX, Brest (FR)

(73) Assignee: Even Sante Industrie, Lesneven (FR)

(21) Appl. No.: 16/067,272

(22) PCT Filed: Dec. 30, 2016
(86) PCT No.: PCT/FR2016/053688

§ 371 Date: Jun. 29, 2018

(30) Foreign Application Priority Data
Dec. 30, 2015 (FR) 1563471

(51) Int. Cl. A23L 33/19; A61K 9/00; A61K 38/17; A23L 33/12; A23L 33/125; A23L 33/16; A23L 33/15; A23L 3/16

(52) U.S. Cl. CPC A23L 33/19; A61K 9/0053; A61K 38/1709; A23L 33/12; A23V 2002/00; A23L 33/16; A23L 33/15; A23L 3/16; A23L 33/125

(57) ABSTRACT

A thermosterilized enteral nutritional composition comprises, per 100 ml of composition, between 8 and 14 g of total proteins, 200 to 350 kcal, the total proteins comprising a quantity of caseinates that is higher than or equal to 55% of the total protein weight of the composition, a quantity of serum proteins that is lower than or equal to 30% of the total protein weight of the composition, and a quantity of caseins that is lower than or equal to 30% of the total protein weight of the composition, the weight ratio of the caseinates to the caseins being higher than or equal to 70/30.

### CROSS-REFERENCE TO RELATED APPLICATIONS

This application is a national phase entry under 35 U.S.C. § 371 of International Patent Application PCT/FR2016/053688, filed Dec. 30, 2016, designating the United States of America and published as International Patent Publication WO 2017/115058 A1 on Jul. 6, 2017, which claims the benefit under Article 8 of the Patent Cooperation Treaty to French Patent Application Serial No. 1563471, filed Dec. 30, 2015.

### TECHNICAL FIELD

This application relates to the field of high-energy-density, protein-rich nutritional compositions for special medical purposes and their method of preparation. These nutritional compositions are intended for the elderly, sick, weakened or those in a state of malnutrition due to illness. These compositions can be used as a main source of supply or as a supplement.

This application relates more specifically to a nutritional hyper-energetic and protein-rich, particularly casein-rich, stable-in-time, single dose and ready-to-use oral nutritional composition and its method of preparation. The composition according to the disclosure is intended for adults and can be used as a primary food source or as a supplement.

### BACKGROUND

The human body is a complex organism. Maintaining vital functions requires energy that is provided through food.

Food provides energy quantifiable in terms of kilojoules or kilocalories.

The proper functioning of the body requires a sufficient and balanced diet. Energy must be provided by proteins, sugars and fats. Proteins are essential for muscle activity, fat is used to store releasable energy based on body needs, and sugars provide an immediate source of energy. These calorie sources are therefore complementary. They must be supplemented by vitamin and mineral intakes that are necessary in the physiological balance of the various vital functions of the body.

This balance can be broken in case of malnutrition due, for example, to an eating disorder (anorexia . . . ), or malnutrition resulting from a pathological situation or in the elderly who no longer feed properly on their own.

Nutritional needs change over one's lifetime. For example, older people have reduced requirements for lipids due to decreased activity and slow cell renewal.

Because of illness, for example, some people are no longer able to eat properly, or even to feed completely. It is therefore necessary to set up supplemental or replacement feeding in order to meet their nutritional needs.

When the digestive tract is still functional, enteral nutrition, i.e., per os or by tube, is preferred. It can be done with nutritional compositions adapted in terms of nutritional profile, viscosity or volume.

For example, some patients cannot ingest large amounts of food. These are, for instance, cachectic patients with or without a pathology, for example, acquired immunodeficiency syndrome, cancer, respiratory or infectious diseases, or trauma.

Protein-rich and hyperenergetic enteral nutritional compositions are known in the art.

Patent Application WO 2014/099795 discloses low viscosity and high caloric density oral nutrition compositions. The compositions of this application mainly comprise non-micellar milk proteins in powder form, in combination with hydrolyzed caseinate.

Patent EP2230940 discloses a high energy density liquid enteral nutritional composition comprising micellar casein, caseinates and optionally a small amount of whey.

Patent Application WO 2002/098242 proposes a high calorie oral liquid supplement whose total proteins consist only of caseinates or a mixture of caseinates and soy proteins.

The solutions of the prior art use proteins that may have lost their native form. The compositions of the prior art whose total proteins consist of milk proteins commonly include a majority of caseins. Such being the case, caseins can react with the acids and other ingredients present in the nutrition composition. These reactions lead to the formation of calcium salts over time resulting in poor stability of the products after packaging.

Solutions of the state of the art have consisted in replacing these caseins with caseinates or mixing these caseins with caseinates, in this case, the caseinates being far fewer than the caseins. Caseinates are more resistant to heat, but they increase the viscosity of the nutritional composition, which may make it more difficult for target patients to consume compositions that are excessively viscous.

Finally, nutritional compositions rich in protein and energy often have organoleptic problems that make them unpalatable to patients. Such being the case, patients likely to consume these nutritional products have little appetite, so it is essential to promote the preservation of good organoleptic properties and a strong palatability.

There is a real need to formulate ready-to-use enteral nutritional compositions with improved, stable, appetizing organoleptic properties while remaining liquid enough to be easily drunk by patients.

### BRIEF SUMMARY

This disclosure proposes to overcome the drawbacks of the prior art by providing an oral, appetizing, stable enteral nutritional composition with improved organoleptic properties that enhances intake by patients with decreased appetite. The composition of this disclosure provides an increased amount of calories and protein while being sufficiently fluid so as to be drunk using a glass or straw.

The enteral nutritional composition is thermosterilized and ready for use.

This disclosure thus relates to an enteral nutritional composition comprising, per 100 ml of composition:
8 to 14 g of total proteins
837 to 1465 kJ (200 to 350 kcal)
total proteins comprising:
   an amount of caseinates greater than or equal to 55% of the total protein weight of the composition,
   an amount of serum proteins greater than or equal to 30% of the total protein weight of the composition,
   an amount of caseins less than or equal to 30% of the total protein weight of the composition,

the weight ratio of the caseinates to the caseins being greater than or equal to 70/30.

It is understood that the total proteins of the enteral nutritional composition according to the disclosure mainly consist of caseinates. The weight ratio of caseinates to caseins is greater than 70/30. It is understood that the proportion of caseinates in relation to caseins is greater than or equal to 70% of the combined weight of caseinates and caseins.

While increasing the proportion of caseinates, however, the inventors had to address the increase in viscosity so that the composition remains drinkable. To do this, they have developed a method of preparation for obtaining a thermosterilized composition with a high proportion of caseinates, especially a weight ratio of caseinates to caseins greater than or equal to 70/30, but whose viscosity remains less than or equal to 800 mPa·s, preferably less than 450 mPa·s when measured at 20° C. to 100 s⁻¹ with a rotary viscometer. This method comprises a sterilization step performed according to the usual methods of Ultra High Temperature, and whose resulting sterilizing value (FO) is greater than or equal to 12.

Thanks to this method, the applicant has surprisingly discovered this particular protein mixture that made it possible to obtain an enteral nutritional composition rich in caseinates that is drinkable, appetizing, stable, with improved organoleptic properties and that enhances intake by patients with decreased appetite.

Advantageously, the caseinates are chosen from sodium caseinates, potassium caseinates, calcium caseinates or a mixture of at least two of these.

In some embodiments, the amount of serum proteins is less than or equal to 25% of the total protein weight of the composition.

In some embodiments, the amount of casein is less than or equal to 25% of the total protein weight of the composition.

Advantageously, the proteins contribute 10% to 40% of the total energy intake per 100 ml of composition.

The viscosity of the composition after thermosterilization is less than or equal to 800 mPa·s when measured at 20° C. to 100 s⁻¹ with a rotary viscometer.

In some embodiments, the viscosity of the composition after thermosterilization is between 30 and 450 mPa·s when measured at 20° C. to 100 s⁻¹ with a rotary viscometer.

Advantageously, the enteral nutritional composition may be used to feed an elderly person, a malnourished person, or a person recovering or suffering from a chronic illness.

### DETAILED DESCRIPTION

A first part of the disclosure consists in composing a thermosterilized enteral nutrition comprising, per 100 ml of composition:
8 to 14 g of total proteins
837 to 1465 kJ (200 to 350 kcal)
total proteins comprising:
   an amount of caseinates greater than or equal to 55% of the total protein weight of the composition,
   an amount of serum proteins less than or equal to 30% of the weight of the total proteins of the composition,
   an amount of caseins less than or equal to 30% of the weight of the total proteins of the composition,

wherein the weight ratio of the caseinates to the caseins is greater than or equal to 70/30,

and for which the viscosity is less than or equal to 800 mPa·s when measured at 20° C. to 100 s⁻¹ with a rotary viscometer.

The composition according to the disclosure can be in various forms depending on the needs of the patients for whom it is intended.

In a particular form of embodiment, the enteral nutritional composition comprises a weight ratio of caseinates/caseins/serum proteins of about 60/25/15.

In a preferred form of embodiment, the enteral nutritional composition comprises:
9 to 12 g of total protein per 100 ml of composition,
an energy density of approximately 1046 kJ (250 kcal) per 100 ml of composition,
a weight ratio of caseinates/caseins/serum proteins of approximately 60/25/15.

According to another form of embodiment, the enteral nutritional composition comprises 8 to 14 g of total proteins per 100 ml of composition.

According to another form of embodiment, the enteral nutritional composition comprises 8 to 12 g of total proteins per 100 ml of composition.

### Definitions

The term “oral” according to this disclosure is understood to mean the nutritional composition is sufficiently fluid to be drunk using a glass or straw. It does not require administration with a spoon. The composition of this disclosure is not intended for tube administration. The viscosity of an “oral” composition according to this disclosure is less than or equal to 800 mPa·s when measured at 100 s⁻¹ to 20° C. with a rotary viscometer. The rotary viscometer is of the cup-and-bob type, for example, a coaxial DIN cylinder. Preferably, the viscosity is between 30 and 600 mPa·s, more preferably between 30 and 450 mPa·s.

The term “thermosterilized” is understood to mean the composition before packaging is sterilized by techniques known to those skilled in the art such as pasteurization and/or sterilization. Preferably, the composition is thermosterilized before aseptic packaging.

The sterilization can be performed by a classic Ultra-High Temperature (UHT) method and known to those skilled in the art. In a preferred form of embodiment, the sterilization is carried out using a UHT method whose resulting sterilizing value (FO) is greater than or equal to 12.

The term “ready to use” is understood to mean the thermosterilized composition is packaged in a bottle, pack, briquette or any other packaging. The composition is packaged in single dose format, generally 50, 100, 125, 150, 200, 250 or 300 ml, preferably 125 ml. The composition thus packaged has a long shelf life, that is to say the “best-before” date (BBD) is at least 6 months, preferably at least 9 months, or even more preferably at least 12 months.

When value ranges are defined by a lower limit and an upper limit, it is understood that the values of the limits are within the intervals.

### Proteins:

The enteral nutritional composition comprises between 8 and 18 grams of protein per 100 ml of composition. Preferably, the composition comprises between 9 and 16 g of proteins per 100 ml, more preferably between 9 and 14 g of proteins per 100 ml of composition. Most preferably, the enteral nutritional composition according to the disclosure comprises between 8 and 12 g or between 9 and 12 g of proteins per 100 ml of composition. The protein comprises at least one source of caseinates, at least one casein source and at least one source of serum proteins (whey). In one form of embodiment, the composition comprises only a milk protein source, provided by cow milk or its derivatives.

The caseinates are provided by sodium caseinates, potassium caseinates, calcium caseinates or a mixture of at least two of these.

The casein is provided by various ingredients that non-exhaustively are Milk Protein Isolates (MPI), Milk Protein Concentrate (MPC), Micellar Casein Isolates (MCI), liquid concentrated milk proteins, skim milk, concentrated skim milk, or a mixture of at least two of these. The casein protein is the constitutive protein of milk casein micelle. Casein may be in micellar or non-micellar form.

The oral enteral nutritional composition comprises an amount of serum proteins less than or equal to 30% of the weight of total proteins of the composition, Serum proteins, i.e., whey proteins, can be provided by total milk proteins (casein sources), serum protein isolates, serum protein concentrates or a mixture of at least two of these. Preferably, the composition comprises less than 25% of serum proteins in relation to the weight of the total proteins of the composition, more preferably less than 20%. In one form of embodiment, the composition comprises about 15% of proteins with respect to the total proteins of the composition.

In one form of embodiment, the combined amount of caseinates and caseins is at least 70% of total protein weight of the composition with a weight ratio of caseinates to caseins greater than or equal to 70/30.

In one form of embodiment, the combined amount of caseinates and serum proteins is at least 60% of total protein weight of the composition with a weight ratio of serum protein caseinates greater than or equal to 60/40.

In a particular form of embodiment, the combined amount of caseinates, caseins and serum proteins is equal to 100% of the total proteins of the composition.

In one form of embodiment, the total proteins of the composition provide between 10% and 40% of the Total Energy Intake (TEI) of the composition. In a preferred form of embodiment, the proteins provide between 12% and 35% of the TEI.

### Carbohydrates:

The enteral nutritional composition further comprises carbohydrates. Carbohydrates provide between 15% and 50% of total energy intake.

Carbohydrates can be simple or complex carbohydrates or a mixture of these. Carbohydrates may include glucose, fructose, sucrose, lactose, trehalose, Palatinose, corn syrup, malt, maltose, isomaltose, partially hydrolyzed maize starch, maltodextrins, glucose syrup, sugar, oligosaccharides, polysaccharides, sweeteners or a mixture of these. Carbohydrates are chosen in order to limit their impact on the viscosity of the nutritional composition as well as to avoid excessive sweetness, excessive Maillard reactions and an excessive osmolarity.

In one form of embodiment for a very fluid enteral nutritional composition, the glucose syrup will be preferred. In another form of embodiment for an oral but less fluid enteral nutritional composition, the carbohydrates may be partially provided by glucose syrup and/or maltodextrin.

In a particular form of embodiment, the glucose syrup and/or maltodextrin may be coupled with another source of carbohydrates.

### Lipids:

The enteral nutritional composition further comprises lipids providing between 20% and 65% of the total energy intakes.

The composition according to the disclosure comprises between 8 and 30 g of lipids per 100 ml of composition.

The lipids used in the composition are animal or vegetable dietary lipids. In one form of embodiment of the disclosure, vegetable oils such as rapeseed, soybean, corn or sunflower oils or a mixture of these, are preferred for their lower cholesterol and/or saturated fatty acid content compared to animal fats.

The combined amount of lipids, carbohydrates and proteins of the composition according to this disclosure is between 35 and 75 g per 100 ml of composition. Preferentially, the combined amount of lipids, carbohydrates and proteins of the composition is between 45 and 65 g per 100 ml of composition.

### Vitamins and Minerals:

The nutritional composition according to the disclosure comprises a large variety of minerals and vitamins to be closer to the recommended daily intake. In one form of embodiment of the disclosure, the nutritional composition has a near-complete to complete nutritional profile in terms of vitamins and minerals according to the European regulation of dietary foods for special medical purposes.

Vitamins and minerals are mostly supplied at least up to 15% of the recommended daily intake defined by European legislation.

### Fibers:

In some forms of embodiments, the composition may be supplemented with dietary fiber. These fibers may be prebiotics such as fructo-oligosaccharides, galacto-oligosaccharides or inulin. The amount of these fibers may be between 0.5 and 6 grams per 100 ml of composition, preferably between 2 and 3 grams per 100 ml of composition. Preferably, the dietary fibers used are soluble. As non-limiting examples, they may be fructans, fructo-oligosaccharides, galacto-oligosaccharides, transgalacto-oligosaccharides, xylooligosaccharides, arabino-oligosaccharides, mano-oligosaccharides, fucooligosaccharides, soy oligosaccharides, inulin or a mixture of these. Insoluble fibers such as cellulose and its derivatives can also be used in addition to soluble fibers.

### Thickeners:

In order to meet the constraints of particular pathologies such as dysphagia, thickeners may be added to the enteral nutritional composition to increase its viscosity.

In a particular form of embodiment, the disclosure may, therefore, comprise thickeners such as starch, vegetable gums, carrageenans, carob, xanthan, guar, pectins, alginates, agar, gelatins or any other thickener known to anyone skilled in the art.

A second part of the disclosure consists of a process for preparing a thermosterilized enteral nutritional composition as defined above, particularly a composition comprising 8 to 14 g of total proteins, in which the weight ratio of caseinates to caseins is higher than or equal to 70/30, and for which the viscosity is less than or equal to 800 mPa·s when measured at 20° C. to 100 s⁻¹ with a rotary viscometer.

Such a method comprises the following steps:
preparation of an aqueous phase,
addition of water, carbohydrates, proteins, minerals and vitamins in the aqueous preparation, addition of fat,
cooling and hydrating the mixture obtained, and
sterilization using a UHT method whose resulting sterilizing value is greater than or equal to 12,

homogenization being performed before or after the sterilization step to stabilize the emulsion.

In alternative procedures, the method may include optional steps such as:
the addition of emulsifiers at the same time as the addition of the fat, or
the addition of dyes and flavors.

In addition, for the purposes of the disclosure, “a sterilizing value greater than or equal to 12” includes a chamber which may range, for example, from 129° C. for 2 minutes to 145° C. for 5 seconds.

### EXAMPLES

The disclosure will be better understood in the light of non-limiting examples of embodiments of the disclosure as described in further detail below. These examples relate to high protein and high calorie beverages that are nutritionally near-complete to complete. The compositions may be used as the sole source of food or as a complement. These are oral supplements belonging to the category of dietary foods for special medical purposes intended to cover the nutritional needs in case of disease-related malnutrition. They may meet current legislation requirements for medical nutrition products.

### Example 1: Method of Preparation and Packaging

According to this disclosure, the composition can be obtained by preparation of an aqueous phase in which are incorporated in one or more steps water, carbohydrates, proteins, minerals, vitamins, dyes, flavors and other additives. The water can be preheated between 30° C. and 60° C. The fat and the emulsifiers can be directly added or prepared separately (fatty phase comprising oils and/or emulsifiers heated and incorporated into the aqueous phase).

A cooling and hydration phase is applied to the mixture thus obtained. The composition is then sterilized according to the usual Ultra-High Temperature methods, with a holding that can go from 129° C. for 2 minutes to 145° C. for 5 seconds; the resulting sterilizing value (Fo) being >=12.

In order to stabilize the emulsion, a homogenization is carried out before or after sterilization, either during the temperature rise phase (heating), or during the cooling before aseptic packaging.

### Example 2: Compositions

Three compositions A, B and C are presented below in Table 1. They are flavored with vanilla, but they can of course be flavored with any other flavor such as chocolate, coffee, strawberry, raspberry, or lemon.

Nutritional compositions are here sterilized by UHT (Ultra-High Temperature) to have a sterilized value of 12 minutes.

The compositions are ready for use and packaged in 125-ml bottles, corresponding to one portion.

These compositions are drinkable with a straw or glass.

Table 1 therefore shows three examples of embodiment of the disclosure. Composition A comprises 9 g of proteins per 100 ml of composition, composition B comprises 10 g of proteins per 100 ml of composition and composition C comprises 12 g of proteins per 100 ml of composition.

TABLE 1
Compositions A, B and C According to the Disclosure

| | per 100 ml | | TEI | | TEI | | TEI |
|---|---|---|---|---|---|---|---|
| Energy | cal | 50 | 00 | 52 | 00 | 51 | 00 |
| Proteins | | | 4 | 0 | 6 | 2 | 9 |
| Casein | | 5% | | 5% | | 5% | |
| (% of total protein weight) | | | | | | | |
| Caseinates | | 0.0% | | 0.0% | | 0.0% | |
| (% of total protein weight) | | | | | | | |
| Serum proteins | | 5% | | 5% | | 5% | |
| (% of total protein weight) | | | | | | | |
| Caseins/caseinates | | 0/70 | | 0/70 | | 0/70 | |
| Lipids | | 0.4 | 8 | 0.2 | 6 | 0.1 | 6 |
| saturated fatty acids | | 0.8 | | 0.8 | | 0.8 | |
| monounsaturated fatty acids | | 0.1 | | 0.1 | | 0.1 | |
| polyunsaturated fatty acids | | 0.9 | | 0.9 | | 0.9 | |
| Carbohydrates | | 0 | 8 | 0 | 8 | 8 | 5 |
| mono-disaccharides | | 0.1 | | 0.1 | | 0.1 | |
| Fibers | Pa · s | 50 | | 24 | | 33 | |
| Viscosity (at 20° C. to 100 s⁻¹) | | | | | | | |

Table 2 describes in more detail a composition D comprising 10 g of proteins per ml of composition. In this composition, the vitamin content is adjusted to cover the nutritional requirements according to the legislation in force.

TABLE 2
Detailed List of Components of a Composition D

| Serving Size | 125 | ml |
|---|---|---|
| Density | 1.145 | g/m L |
| Composition for 100 ml | | |
| Energy | 250 | kcal |
| PROTEINS | 10 | g |
| Casein (%/proteins) | 24.0% | |
| Caseinates (% of proteins) | 59.0% | |
| Serum proteins (% of proteins) | 17.0% | |
| Serving Size | 125 | ml |
| Casein/caseinate | 29/71 | |
| LIPIDS | 10 | g |
| saturated fatty acids | 0.8 | g |
| monounsaturated fatty acids | 6.1 | g |
| polyunsaturated fatty acids | 2.9 | g |
| linoleic acid | 2.03 | g |
| w6/w3 | 2.3 | g |
| CARBOHYDRATES | 30 | g |
| mono-disaccharides | 6.2 | g |
| polysaccharides | 24 | g |
| sucrose | 1.5 | g |
| lactose | <0.5 | g |
| MINERALS | | |
| Calcium | 150 | mg |
| Copper | 350 | mg |
| Iron | 1.5 | mg |
| Potassium | 240 | mg |
| Magnesium | 25 | mg |
| Manganese | 0.36 | mg |
| Sodium | 95 | mg |
| Phosphorus | 160 | mg |
| Zinc | 1.6 | mg |
| Chromium | 15 | mg |
| Chloride | 100 | mg |
| Fluorine | 0.01 | mg |
| Molybdenum | 14 | mg |
| Iodine | 4 | mg |
| Selenium | 14 | mg |

Table 3 describes a composition E comprising 10 g of proteins per ml and in which the caseinate/casein ratio is equal to 100/0. The vitamin content is adjusted to cover the nutritional requirements according to the legislation in force.

TABLE 3
Detailed List of Components of a Composition E

| Serving Size | 125 | ml |
|---|---|---|
| Serving Size | 125 | ml |
| Density | 1.145 | g/m |
| Composition for 100 ml | | |
| Energy | 250 | kcal |
| PROTEINS | 10 | g |
| Casein (%/proteins) | 0 | |
| Caseinates (% of proteins) | 88% | |
| Serum proteins (% of proteins) | 12% | |
| Casein/caseinate | 0/100 | |
| LIPIDS | 10 | g |
| saturated fatty acids | 0.8 | g |
| monounsaturated fatty acids | 6.1 | g |
| polyunsaturated fatty acids | 2.9 | g |
| linoleic acid | 2.03 | g |
| w6/w3 | 2.3 | g |
| CARBOHYDRATES | 30 | g |
| mono-disaccharides | 6.2 | g |
| polysaccharides | 24 | g |
| sucrose | 1.5 | g |
| lactose | <0.5 | g |
| MINERALS | | |
| Calcium | 92 | mg |
| Copper | 350 | mg |
| Iron | 1.5 | mg |
| Potassium | 92 | mg |
| Magnesium | 22 | mg |
| Manganese | 0.5 | mg |
| Sodium | 95 | mg |
| Phosphorus | 130 | mg |
| Zinc | 1.6 | mg |
| Chromium | 15 | mg |
| Chloride | 110 | mg |
| Fluorine | 0.01 | mg |
| Molybdenum | 14 | mg |
| Iodine | 4 | mg |
| Selenium | 14 | mg |

1.-10. (canceled)

11. A thermosterilized enteral nutritional composition, comprising per 100 ml of the composition:
8 to 14 g of total proteins; and
837 to 1465 kJ (200 to 350 kcal); and
wherein the total proteins comprise:
an amount of caseinates greater than or equal to 55% of the total protein weight of the composition;
an amount of serum proteins greater than or equal to 30% of the total protein weight of the composition; and
an amount of caseins less than or equal to 30% of the total protein weight of the composition;
wherein a weight ratio of the caseinates to the caseins is greater than or equal to 70/30; and
wherein a viscosity of the composition is less than or equal to 800 mPa·s when measured at 20° C. to 100 s⁻¹ with a rotary viscometer.

12. The enteral nutritional composition of claim 11, wherein the amount of serum proteins is less than or equal to 25% of the total protein weight of the composition.

13. The enteral nutritional composition of claim 11, wherein the amount of casein is less than or equal to 25% of the total protein weight of the composition.

14. The enteral nutritional composition of claim 11, wherein the proteins contribute 10% to 40% of the total energy intake per 100 ml of composition.

15. The enteral nutritional composition of claim 11, wherein the caseinates are selected from sodium caseinates, potassium caseinates, calcium caseinates or a mixture of at least two of these.

16. The enteral nutritional composition of claim 15, wherein the amount of serum proteins is less than or equal to 25% of the total protein weight of the composition.

17. The enteral nutritional composition of claim 16, wherein the amount of casein is less than or equal to 25% of the total protein weight of the composition.

18. The enteral nutritional composition of claim 17, wherein the proteins contribute 10% to 40% of the total energy intake per 100 ml of composition.

19. The enteral nutritional composition of claim 11, wherein the composition comprises:
9 to 12 g of total protein per 100 ml of composition;
an energy density of approximately 1046 kJ (250 kcal) per 100 ml of composition; and
a weight ratio of caseinates/caseins/serum proteins of approximately 60/25/15.

20. The enteral nutritional composition of claim 19, wherein a viscosity of the composition is between 30 and 450 mPa·s when measured at 20° C. to 100 s⁻¹ with a rotary viscometer.

21. An enteral nutritional composition according to claim 11 for feeding an elderly person, a malnourished person, a convalescent or a chronically ill person.

22. A method for the preparing the enteral nutritional composition of claim 1, comprising the following steps:
preparation of an aqueous phase;
addition of water, carbohydrates, proteins, minerals and vitamins in the aqueous preparation phase;
addition of fat;
cooling and hydration of the mixture obtained; and
sterilization of the mixture using a UHT method whose resulting sterilizing value is greater than or equal to 12, a homogenization being performed before or after the sterilization step in order to stabilize an emulsion.

23. The method of claim 22, further comprising adding one or more components selected from the group consisting of emulsifiers, which may be added at the same time as the fat, colorants, and flavors.
